(19) United States
(12) Patent Application Publication
(10) Pub. No.: US 2010/0114152 A1
(43) Pub. Date: May 6, 2010

(54) Minimally-Invasive Method and Device for Permanently Compressing Tissues within the Body

(76) Inventor: Himanshu SHUKLA, Scottsdale, AZ (US)

Correspondence Address: ETHERTON LAW GROUP, LLC, 2010 E. University Drive, Suite 25, Tempe, AZ, 85281, US

(21) Appl. No.: 12/395,870

(22) Filed: Mar. 2, 2009

Related U.S. Application Data
(60) Provisional application No. 61/198,545, filed on Nov. 6, 2008.

(51) Int. Cl. A61M 29/00

(52) U.S. Cl. 606/198

(57) ABSTRACT

The present invention is method for permanently compressing tissues in the body. The method employs a compression device made of a spring and a flexible sheet that cooperate to form a compressive envelope around the desired tissue. The spring is preferably Z-shaped or a coil. The sheet is made of a flexible material and the material is preferably elastic. The sheet is preferably a biocompatible elastic material, such as a mesh made of stainless steel or a woven or non-woven elastomer. The method is minimally invasive because it deploys the compression device through the patient's skin directly to the tissue, as opposed to through catheterization or open invasive surgery, such as open-heart surgery. The preferred use is for compressing the left atrial appendage to prevent clots from forming and circulating, thereby preventing strokes. The device is deployed by making an incision in a patient's chest, inserting the compression device through the incision into the pericardium without piercing the heart, and deploying it around the entire appendage. The device remains in place by its own compressive nature: either the spring, the sheet, or both components compress the appendage and cause the device to stay in place due to friction. Preferably the entire compression device is left in place, but the spring or the sheet may be removed, leaving the remaining component to compress the appendage.

### CROSS-REFERENCE TO RELATED APPLICATIONS

This application claims the benefit of co-pending provisional application No. 61/198,545 filed Nov. 6, 2008.

### FIELD OF INVENTION

This invention relates to surgery methods and devices. This invention relates particularly to methods and devices for collapsing and occluding appendages and tissues within the body, including the left atrial appendage.

### BACKGROUND

An arrhythmia is the irregular beating of the heart which limits the ability of the heart to pump blood effectively. It is caused when the natural rhythm of the heart becomes abnormal, the regular pathways for electrical signals in the heart are interrupted, or parts of the heart improperly emit electrical signals. Arrhythmias vary in seriousness, from brief, almost imperceptible events, to life-threatening conditions.

Atrial fibrillation is one form of arrhythmia which affects over two million people. Atrial fibrillation is a serious disease which can lead to chronic fatigue, congestive heart failure, and stroke. Atrial fibrillation occurs when the atria pump quickly and unevenly. This quivering creates a serious risk that blood will collect and pool in the left atrial appendage. If this pooled blood forms clots that leave the heart, a stroke can occur.

A number of methods have been used to treat arrhythmias. Blood thinners can be used to lower the risk of clotting in atrial fibrillation patients. Antiarrhythmic drugs can reduce the abnormal firing of electrical impulses in the heart. However, medication must be taken regularly and frequently is accompanied by unwanted side effects. Defibrillation can be performed in an emergency situation when the heart has departed from its normal rhythm. In some circumstances, a cardioverter defibrillator may be implanted in the patient's chest to shock the heart back into rhythm.

Surgical methods are known in which portions of the heart can be ablated to kill cells that emit extra impulses. Methods for occlusion of heart tissue have also been developed; these techniques frequently focus on isolating the left atrial appendage by ligating it at its base. This often involves surgically opening the chest cavity, which significantly increases the risk to the patient and adds significant recovery time. Other methods involve endocardial approaches, in which a catheter is inserted through the femoral or jugular vein into the heart to deliver devices that occlude or remove the left atrial appendage. While reducing the patient's recovery time, open surgery and entering the heart from within still creates significant risk to the patient.

Non-invasive surgical methods are known for examining or repairing certain tissues. For example, arthroscopic surgery is a procedure performed through small incisions in the skin to repair injuries to tissues such as ligaments, cartilage, or bone within a joint area. The surgery is conducted with the aid of an endoscope, a small instrument guided by a lighted scope attached to a television monitor. Other instruments are inserted through additional small incisions around the knee, advantageously requiring less anesthetic, less cutting, and less recovery time than invasive surgery.

It is desirable to minimize or eradicate atrial fibrillation without opening the chest cavity or entering the interior of the heart. Therefore, it is an object of this invention to provide minimally-invasive surgical methods and devices to compress tissue inside the body. It is another object to compress the left atrial appendage to prevent blood from pooling and forming clots. It is another object to provide a compression device that remains in place without sutures or ancillary devices. It is another object to provide compression of the atrial appendage that is essentially permanent.

### SUMMARY OF THE INVENTION

The present invention is method for permanently compressing tissues in the body. The method employs a compression device made of a spring and a flexible sheet that cooperate to form a compressive envelope around the desired tissue. The spring is preferably Z-shaped or a coil. The sheet is made of a flexible material and the material is preferably elastic. The sheet is preferably a biocompatible elastic material, such as a mesh made of stainless steel or a woven or non-woven elastomer. The method is minimally invasive because it deploys the compression device through the patient's skin directly to the tissue, as opposed to through catheterization or open invasive surgery, such as open-heart surgery. The preferred use is for compressing the left atrial appendage to prevent clots from forming and circulating, thereby preventing strokes. The device is deployed by making an incision in a patient's chest, inserting the compression device through the incision into the pericardium without piercing the heart, and deploying it around the entire appendage. The device remains in place by its own compressive nature: either the spring, the sheet, or both components compress the appendage and cause the device to stay in place due to friction. Preferably the entire compression device is left in place, but the spring or the sheet may be removed, leaving the remaining component to compress the appendage.

### BRIEF DESCRIPTION OF THE DRAWINGS

FIG. 1 is a side view of an untreated heart.

FIG. 2 is a side view of a heart with the compression device of FIG. 4 in place around the left atrial appendage.

FIG. 3a is a top view of the preferred embodiment of the compression device using a Z-spring.

FIG. 3b is an end view of the preferred embodiment of the compression device using a Z-spring, as the spring portions are squeezed towards each other to form an aperture through which the tissue is inserted.

FIG. 4 is a top view of a second embodiment of the compression device using a modified Z-spring.

FIG. 5 is a top view of a third embodiment of the compression device using a modified Z-spring.

FIG. 6 is a top view of an embodiment of the compression device using a coil as the spring.

FIGS. 7a-e show several types of flexible materials used as part of the compression device.

FIG. 8 is a cross-sectional view of an insertion tool that pushes the compression device onto the appendage.

FIG. 9 is a cross-sectional view of another insertion tool that pushes the compression device onto the appendage

### DETAILED DESCRIPTION OF THE INVENTION

The present invention is method for permanently compressing tissues within the body so that they no longer function or no longer cause dysfunction. The method employs a compression device 10 that envelopes substantially the entire tissue. The method may be applied to tissues in the body such the appendix, gallbladder and stomach, and will be described with particular reference to the left atrial appendage to prevent blots clots from forming inside it.

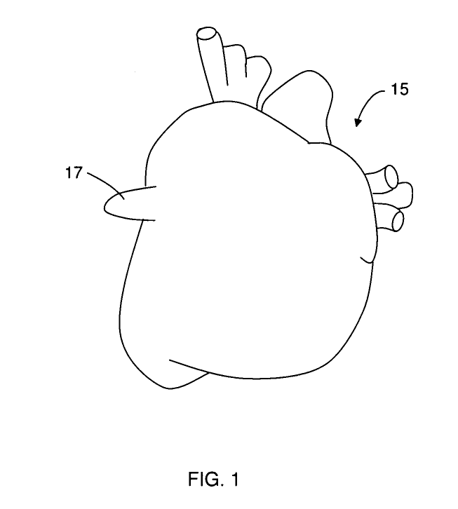

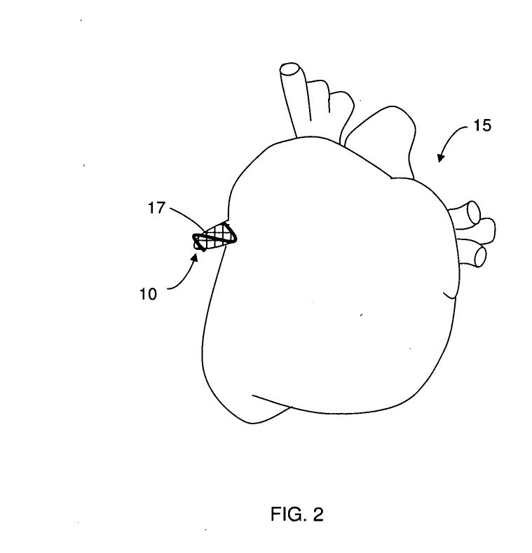

FIG. 1 shows an untreated heart 15 and left atrial appendage 17 (referred to hereafter as “LAA”). FIG. 2 shows a heart 15 after being treated with the method herein, namely with the preferred embodiment of the compression device 10 disposed around the LAA 17.

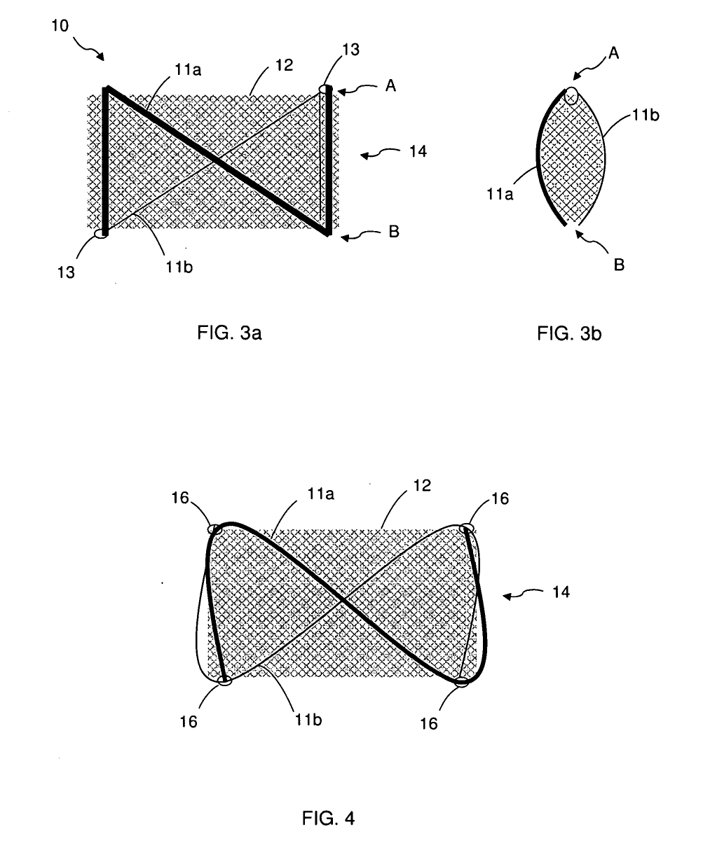

FIGS. 3-6 show several embodiments of the compression device 10. Each compression device 10 comprises a spring 11 and a flexible sheet 12. The spring 11 can take any form provided that in its relaxed state the opposing sides of the spring are biased towards each other, thus tending to compress anything between the opposing sides. In FIG. 3 the spring 11 is comprised of two Z-shape portions of metal 11a and 11b. Spring portion 11a is one side of the compression device 10 and spring portion 11b is on the opposing side; the spring portions may be connected or separate. FIG. 3 shows a single piece of metal formed into a Z, wound back upon itself at loops 13. Portions 11a and 11b tend to return to their original shape and orientation after being deformed. Other materials can be used for the spring and its elasticity can be provided by the shape of the spring or the material it is made of, or both.

The preferred embodiment of the compression device 10 is substantially planar when at rest. When the spring portions 11a and 11b are squeezed towards each other at the points of the Zs indicated in FIG. 3 as A and B, the portions deform apart from each other such that an aperture is formed between the opposing sides of the compression device 10. See FIG. 3b which shows the end view of the device in FIG. 3a after being deformed. This deformed state is referred to herein as the “open” state. In some embodiments of the invention, the aperture may take on a cross-sectional shape that may be elliptical like that shown in FIG. 3b, or round, square, rectangular, oval, triangular, star-shaped, etc. When the compression device is in the open state it can be pushed onto the LAA like putting a mitten on a hand. Portions of the spring 11 that form the leading edge 14 of the compression device 10 provide structural rigidity relative to the LAA so that the compression device does not deform much, if at all, when its leading edge abuts the LAA.

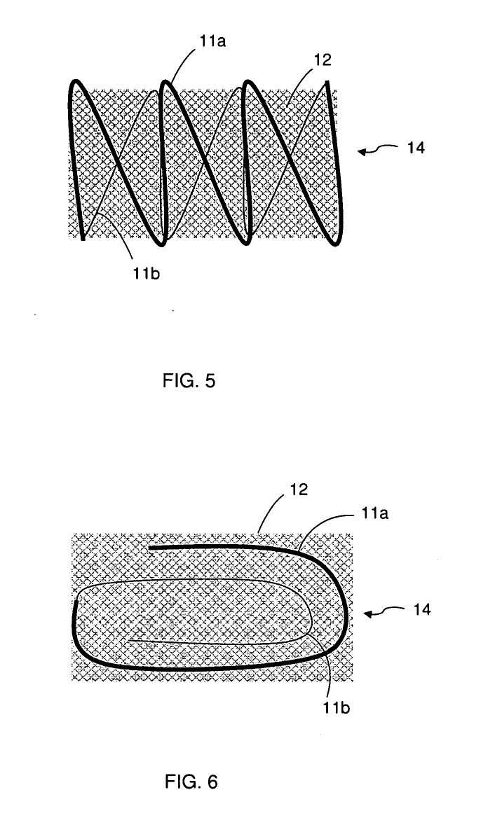

FIG. 4 shows a second embodiment of the compression device 10 employing a modified Z-spring having softer angles at the points of the Zs, which may avoid tissue damage that sharper points could induce. Spring portion 11a is one side of the compression device 10 and spring portion 11b is on the opposing side; the spring portions are separate but operatively connected at rings 16 so that an aperture is formed between the opposing sides of the compression device 10 when spring portion 11a and 11b are squeezed towards each other. FIG. 5 shows a third embodiment of the compression device 10 employing a single piece of metal formed into a Z, wound back upon itself a number of times. In FIG. 6 the spring 11 is a coil shaped much like a paper clip. The portion or portions of the spring 11 that forms the leading edge 14 provides structural rigidity relative to the LAA. In contrast to the Z shape, however, the embodiment in FIG. 6 is deformed into the open state by forcing the LAA between spring portion 11a and spring portion 11b, much like placing a paper clip over the edge of a piece of paper. In its relaxed state the opposing sides of the spring 11 are biased towards each other, thus tending to compress anything between the opposing sides.

The spring 11 is operatively connected to a flexible sheet 12. Spring portion 11a is connected to one side of the sheet 12 and spring portion 11b is operatively connected to the opposing side of the sheet 12. The sheet 12 is flexible and cooperates with the spring such that when the spring portions deform apart from each to form the aperture, the sheet also deforms and forms a pouch for receiving the appendage. The sheet 12 is also preferably elastic, so that it tends to compress any tissue inside the pouch.

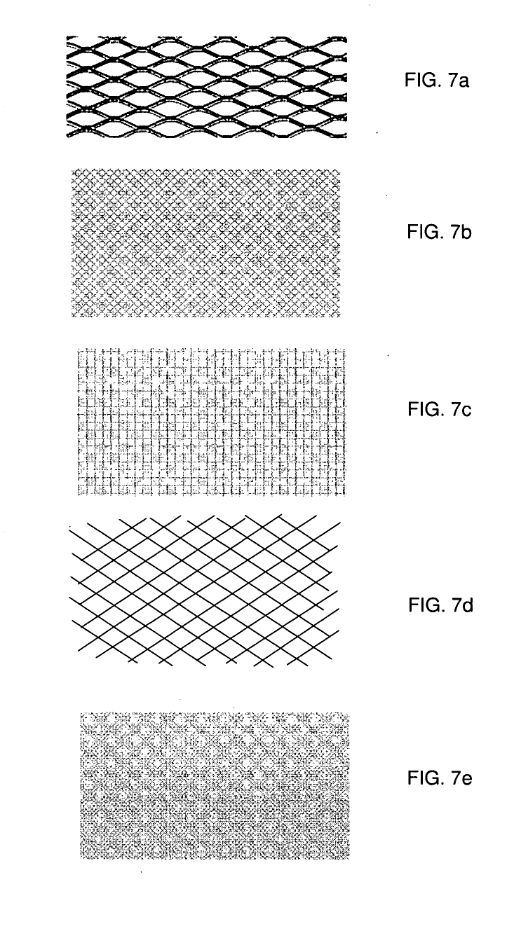

The sheet 12 is preferably made from a biocompatible, elastic material such as a metal wire mesh. The density, shape, and size of the holes in the mesh will depend on the material properties desired for the sheet 12 and the material it is made of. Woven and non-woven materials can be used for the sheet 12. FIG. 7a illustrates a mesh of expanded material, much like expanded metal comprising bonds and strands. FIG. 7b illustrates a fine mesh, and FIGS. 7c and d illustrate less fine mesh. FIG. 7e illustrates a woven material wherein the size of the holes between the weft and warp is extremely small relative to the diameter of the thread. Other acceptable materials include silicon rubber, polyurethane, super-elastic material, shape-memory polymer or metal such as nitriol, latex, nitrile, butyl, styrene-butadiene, polyacrylate, acrylic, polyisoprene, chloroprene, and fluoroelastomers. The sheet 12 may also incorporate pharmacological agents, sensors, smart materials, and materials that are observable with electromagnetic energy, such as with ultrasound or radio frequencies.

The size of the compression device 10 will depend on a number of factors, including the amount of tissue to be compressed, how elastic the sheet is, the compressive force provided by the spring and sheet, etc.

The compression device 10 is implanted using minimally-invasive surgical procedures to minimize trauma to the patient. In general an endoscope 81 is inserted through a tiny incision near the tissue to be collapsed. The camera relays images to a computer screen. The surgeon uses the images to guide other small instruments to the desired location, which may be inserted through the same incision or through additional incisions. To insert the compression device onto the LAA, a needle is inserted into the subxiphoid area of the patent and into the pericardial space. A guidewire is then advanced into the pericardial space, and the needle is removed. A sheath is placed around the guidewire and inserted into the pericardial space. The compression device is inserted inside the sheath into the pericardial space and around the LAA.

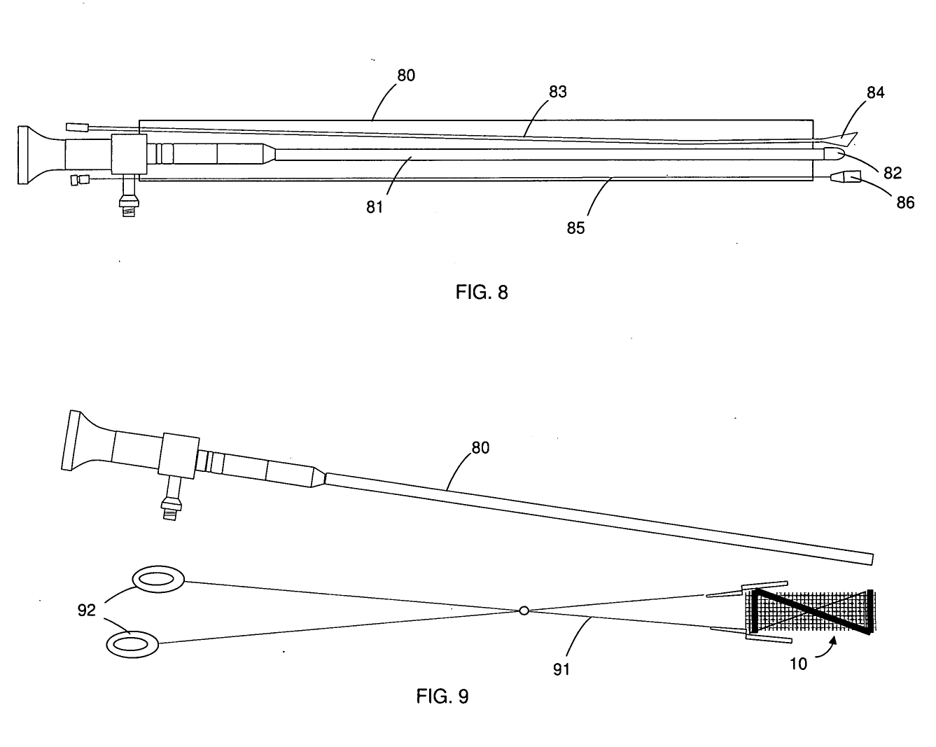

In one embodiment, the endoscope 81 and the compression device 10 are inserted into the body through the same sheath 80. See FIG. 8. The endoscope 81 has a transparent balloon 82 attached to its end which can be inflated against the surface of the heart, allowing a landscape view of its structure. While the balloon 82 is inflated, a large-bore suction tube 83 is placed through the sheath and its end, preferably a suction cup 84, is pressed against the LAA (not shown). The suction cup 84 then grasps the LAA and the compression device (not shown) is advanced over the suction tube 83 to the base of the appendage.

FIG. 9 shows a different insertion device. In this example, the compression device is manipulated separately from the endoscope. Again, the endoscope 81 is inserted into the body through a small incision in the patient's skin. Tongs 91 retaining the compression device 10 are also inserted into the body through a small incision in the patient's skin, through the same or a different incision as the endoscope. The tongs 91 are guided to the LAA, and the compression device 10 is placed over the LAA by squeezing the tongs 91, such that the spring portions 11a and 11b are squeezed towards each other to open the compression device. The compression device is guided over the LAA and, upon desired placement, the tongs are released, allowing the compression device to compress around the LAA. The compression device may be repositioned as necessary during surgery, but is eventually left in place to permanently compress the LAA. Alternatively, either the spring or sheet, or both, may be made of materials that are designed to degrade in the body over time. This is advantageous when, having been compressed long enough for the sides of LAA to have grown together, the LAA stays compressed by its own accord.

The device is retained in place by its own compressive nature: either the spring, the sheet, or both components compress the appendage and cause the device to stay in place. The friction between the compression device 10 and the LAA increases when the tissue bulges out from the holes of the mesh. Preferably the entire compression device is left in place, but the spring or the sheet may be removed, leaving the remaining component to compress the appendage.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims.

I claim:

1. A method for compressing tissue inside a patient's body, the method comprising:
a) disposing a compression device around the tissue wherein the compression device comprises:
i. a spring; and
ii. a flexible sheet cooperating with the spring;
b) providing compression of the tissue using the compressive nature of the compression device; and
c) retaining the compression device around the tissue using the compressive nature of the compression device.

2. The method of claim 1 wherein retaining the compression device around tissue is caused by the compressive nature of the spring.

3. The method of claim 2 further comprising removing the sheet form the patient's body.

4. The method of claim 1 wherein retaining the compression device around tissue is caused by the compressive nature of the sheet.

5. The method of claim 4 further comprising removing the spring form the patient's body.

6. The method of claim 1 wherein the tissue is the left atrial appendage.

7. The method of claim 6 compression device envelopes substantially the entire left atrial appendage.

8. A method for compressing the left atrial appendage comprising:
a) making an incision in a patient's chest;
b) inserting a compression device through the incision into the pericardium without piercing the heart, wherein the compression device comprises:
i. a spring; and
ii. a flexible sheet cooperating with the spring;
c) disposing the compression device around the left atrial appendage;
d) providing compression of the left atrial appendage by the compressive nature of the compression device; and
e) closing the incision.

9. The method of claim 8 further comprising retaining the compression device around the left atrial appendage using the compressive nature of the spring.

10. The method of claim 9 further comprising removing the sheet from the patient's body.

11. The method of claim 8 further comprising retaining the compression device around the left atrial appendage using the compressive nature of the sheet.

12. The method of claim 11 further comprising removing the spring from the patient's body.

13. A device for compressing tissue inside a patient's body, the device comprising:
a) a spring; and
b) a flexible sheet cooperating with the spring.

14. The device of claim 13 wherein the tissue is a left atrial appendage.

15. The device of claim 13 wherein the spring is substantially Z-shaped.

16. The device of claim 13 wherein the spring is substantially a coil.

17. The device of claim 13 wherein the sheet is mesh.

18. The device of claim 13 wherein the sheet is nitinol.

19. The device of claim 13 wherein the sheet is elastic.

20. The device of claim 13 wherein the spring or sheet degrades in the body over time.
